(19) United States
(12) Patent Application Publication
(10) Pub. No.: US 2019/0353740 A1
(43) Pub. Date: Nov. 21, 2019

(54) METHOD AND APPARATUS FOR MAGNETIC RESONANCE ANGIOGRAPHY

(71) Applicant: Siemens Healthcare GmbH, Erlangen (DE)

(72) Inventor: Qiong Zhang, Shenzhen (CN)

(73) Assignee: Siemens Healthcare GmbH, Erlangen (DE)

(21) Appl. No.: 16/415,415

(22) Filed: May 17, 2019

(30) Foreign Application Priority Data
May 17, 2018 (CN) 201810473854.X

(51) Int. Cl. G01R 33/563; G01R 33/54; G01R 33/561; G01R 33/48; A61B 5/055; G06T 11/00

(52) U.S. Cl. CPC G01R 33/5635; G01R 33/543; G01R 33/5611; G06T 2211/404; A61B 5/055; G06T 11/003; G01R 33/4818

(57) ABSTRACT

In a method and apparatus for magnetic resonance angiography (MRA) a time of flight (TOF) scanning sequence is used that has a first TOF subsequence having at least a saturated zone (T-sat) module and at least an excitation module, with a T-sat module directly followed by at least an excitation module, and a second TOF subsequence that has at least an excitation module and no T-sat module. K-space data, as a first k-space data portion are acquired after each excitation module of the first TOF subsequence, with the first k-space data portion filling the central area of k-space. K-space data, as a second k-space data portion are acquired after each excitation module of the second TOF subsequence, with the said second k-space data portion filling the edge area of k-space. The k-space data are used to reconstruct a magnetic resonance image.

### BACKGROUND OF THE INVENTION

### Field of the Invention

The present invention concerns a method, apparatus and non-transitory data storage medium for magnetic resonance angiography (MRA).

### Description of the Prior Art

MRA (Magnetic Resonance Angiography) is a technique using magnetic resonance to display blood vessels. A TOF (Time-Of-Flight) sequence can usually be adopted in the methods for MRA. A TOF sequence usually comprises a plurality of T-sat modules and a plurality of excitation modules, and a T-sat module is followed by an excitation module. Wherein, each T-sat module comprises a T-sat radio frequency (RF) pulse and a gradient pulse, and each excitation module comprises an excitation RF pulse and a gradient pulse. Before each excitation RF pulse, a TOF sequence uses T-sat RF pulses plus a spoiled gradient to be saturated parallel to the angiographic plane and all spin signals located in an area at the distal end, including signals of venous blood in the area. This method can well suppress signals of venous blood flowing into the angiographic plane during angiography. However, since the power of a T-sat RF pulse is high and the duration is long, such a magnetic resonance imaging method is time consuming and the imaging efficiency is low. In addition, dense T-sat RF pulse recurrences will also lead to a high specific absorption rate (SAR).

### SUMMARY OF THE INVENTION

An object of the present invention is to provide a method and apparatus for MRA wherein the above-problems are avoided, or at least reduced.

The method for MRA provided by the present invention includes scanning (i.e., acquiring MRA data by executing) a TOF sequence, wherein the TOF sequence includes a first TOF subsequence and a second TOF subsequence. The first TOF subsequence includes at least a T-sat module and at least an excitation module. The T-sat module is directly followed by at least an excitation module, and the second TOF subsequence includes at least an excitation module. Some acquired data are entered into a memory as k-space data in a first k-space data portion after each excitation module of the first TOF subsequence, in order to fill the first k-space data portion in the central area of k-space. Other acquired data are entered into the memory as k-space data in a second k-space data portion after each excitation module of the second TOF subsequence, in order to fill the second k-space data portion in the edge area of k-space. The k-space data after filling are used to reconstruct a magnetic resonance image.

Through this method, k-space is divided into a central area and an edge area. As is known, data entered in the central area has the greatest effect on the imaging result, so k-space data excited by a first TOF subsequence composed of T-sat modules are entered into this central region so as to produce the best imaging effect. For the edge area, which affects the imaging result less, k-space data excited by the second TOF subsequence having no T-sat module are acquired so as to reduce the total number of T-sat modules used for the TOF sequence. Thus, the scanning time is reduced accordingly, the scanning efficiency is improved, and the SAR is reduced.

In an embodiment of the present invention, the magnetic resonance blood vessel scanning further includes setting a percentage of the central area of k-space, wherein the ratio of the number of excitation modules of the first TOF subsequence to the total number of excitation modules of the TOF sequence is equal to the percentage of the central area of k-space. This percentage of the central area of k-space is adjustable.

In this embodiments of the present application, the percentage of the central area of k-space can be set according to experience and can vary with detected parts and detected persons. In addition, the percentage of the central area of k-space is adjustable so that a tradeoff is obtained between the reduction of the image effect and the reductions of the scanning time and the SAR. In other words, while the imaging effect is assumed, the percentage of the central area of k-space is reduced as much as possible so as to reduce the scanning time and the SAR as much as possible.

In another embodiment of the present invention, the magnetic resonance blood vessel scanning method further includes setting a TOF sequence segmentation value N, wherein the T-sat module is directly followed by N excitation modules and N is a positive integer. This TOF sequence segmentation value N is adjustable.

The larger N is, the more the scanning time and the SAR are reduced. Of course, N also affects the imaging effect. Usually, the smaller N is, the better the imaging effect. Therefore, a tradeoff can also be obtained between the reduction of the imaging effect and the reductions of the scanning time and the SAR by adjusting N. In other words, while the imaging effect is assumed, the percentage of the central area of k-space is reduced as much as possible so as to reduce the scanning time and the SAR as much as possible.

In another embodiment of the present invention, acquiring k-space data after each excitation module of the first TOF subsequence includes using a rapid parallel acquisition technique so as to acquire the k-space data after each excitation module of the first TOF subsequence, and acquiring k-space data after each excitation module of said second TOF subsequence includes using the rapid parallel acquisition technique so as to acquire the k-space data after each excitation module of the second TOF subsequence.

In addition, the scanning time can further be shortened and the SAR can further be lowered by combining other rapid k-space data acquisition techniques with the technical solution in the present application.

An MRA apparatus according to the present invention has an MRA data acquisition scanner that is operated by a computer in order to scan a TOF sequence, wherein THE TOF sequence includes a first TOF subsequence and a second TOF subsequence. The first TOF subsequence includes at least a T-sat module and at least an excitation module. The T-sat module is directly followed by at least an excitation module. The said second TOF subsequence includes at least an excitation module.

The computer is configured to acquire k-space data as a first k-space data portion after each excitation module of said first TOF subsequence and to acquire k-space data as a second k-space data portion after each excitation module of said second TOF subsequence.

The computer is configured to fill said first k-space data portion in the central area of k-space and fill said second k-space data portion in the edge area of said k-space.

An image reconstruction computer, configured to use the k-space data after filling to reconstruct a magnetic resonance image.

In the inventive MRA apparatus, k-space is divided into a central area and an edge area. As noted above, the central area has the greatest affect on the imaging quality, so k-space data excited by the first TOF subsequence including T-sat modules are entered into the central area so as to assure good image quality. The edge area, which affects the imaging result less, is filled with k-space data excited by the second TOF subsequence have no T-sat module, so as to reduce the total number of T-sat modules used for a TOF sequence. Thus, the scanning time is reduced accordingly, the scanning efficiency is improved, and the SAR is reduced.

In an embodiment of the present invention, the MRA apparatus further has a setting processor, configured to set a percentage of the central area of k-space, wherein the ratio of the number of excitation modules of the first TOF subsequence to the total number of excitation modules of the TOF sequence is equal to the percentage of the central area of k-space.

The percentage of the central area of k-space can be set according to the experience and can vary with detected parts and detected persons. In addition, the percentage of the central area of k-space is adjustable so that a tradeoff is obtained between the reduction of the image effect and the reductions of the scanning time and the SAR. In other words, while the imaging effect is assured, the percentage of the central area of k-space is reduced as much as possible so as to reduce the scanning time and the SAR as much as possible.

In an embodiment of the present invention, the setting processor is further configured to set a TOF sequence segmentation value N, wherein the T-sat module is directly followed by N excitation modules and N is a positive integer.

A tradeoff can also be obtained between the reduction of the imaging effect and the reductions of the scanning time and the SAR by adjusting N. In other words, while the imaging effect is assured, the percentage of the central area of k-space is reduced as much as possible so as to reduce the scanning time and the SAR as much as possible.

In an embodiment of the present invention, the computer operates the scanner using a rapid parallel acquisition technique to acquire the k-space data.

In addition, the scanning time can further be shortened and the SAR can further be lowered by combining other rapid k-space data acquisition techniques with the technical solution in the present application.

The present invention also encompasses a non-transitory, computer-readable data storage medium encoded with programming instructions that, when the storage medium is loaded into a computer of an MRA apparatus, cause the computer to operate the MRA apparatus so as to implement any or all embodiments of the method according to the invention, as described above.

### BRIEF DESCRIPTION OF THE DRAWINGS

FIG. 1 is a flowchart of the method for MRA according to an embodiment of the present invention.

FIG. 2 is a schematic diagram for a TOF sequence according to an embodiment of the present invention.

FIG. 3 is a schematic diagram for a TOF sequence according to another embodiment of the present invention.

FIG. 4a is a schematic diagram illustrating k-space filling according to an embodiment of the present invention.

FIG. 4b is a schematic diagram illustrating k-space filling according to another embodiment of the present invention.

FIG. 4c is a schematic diagram illustrating k-space filling according to a further embodiment of the present invention.

FIG. 5 is a block diagram of the basic components of the apparatus for MRA according to an embodiment of the present invention.

FIG. 6 is a schematic diagram for the hardware structure of the apparatus for MRA according to one embodiment of the present invention.

FIG. 7a shows the imaging result produced by executing a conventional TOF sequence scan.

FIG. 7b shows an imaging result produced using the method in the embodiment of the present application, wherein the percentage of the central area of k-space is 50% and N=1.

FIG. 7c shows another imaging result produced using the method in the embodiment of the present application, wherein the percentage of the central area of k-space is 50% and N=2.

### DESCRIPTION OF THE PREFERRED EMBODIMENTS

To make clearer the object, technical solutions and advantages of the present invention, the following describes the technical solutions in the embodiments of the present invention in combination with the drawings for the embodiments of the present invention. The embodiments described herein are only representative of all possible embodiments of the present invention. Other embodiments will be understood by those skilled in the art to be within the scope of the present invention.

The terms “comprise” and “have” and their variants in the description and claims of the present invention are intended to cover non-exclusive inclusion. For example, the process or method comprising a series of steps or the system, product or equipment comprising a series of units are unnecessarily limited to those listed steps or units, but can comprise other steps or units which are not clearly listed or are intrinsic to the process, method, product or equipment.

As described above, a TOF sequence is widely applied in MAR, but the problems such as a long scanning time, a low imaging efficiency and a high SAR exist in the application of a TOF sequence.

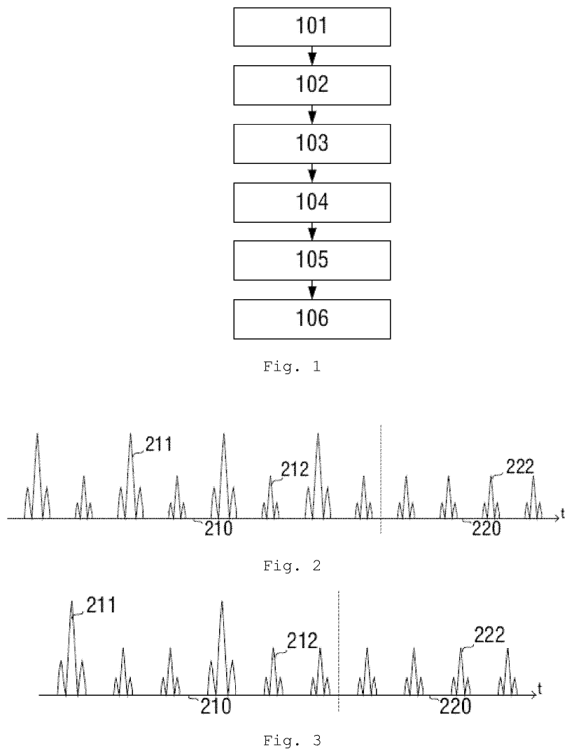

FIG. 1 is a flowchart of the method for MRA according to an embodiment of the present application. As shown in FIG. 1, the method for MRA described in the embodiment of the present application includes the following steps:

Step 101: Scan a TOF sequence.

In the embodiments of the present application, said TOF sequence comprises a first TOF subsequence and a second TOF subsequence. Wherein, the first TOF subsequence includes at least a T-sat module and at least an excitation module, and a T-sat module is directly followed by at least an excitation module. The second TOF subsequence includes at least an excitation module, but no T-sat module.

FIG. 2 is a schematic diagram for a TOF sequence according to one embodiment of the present application. As shown in FIG. 2, the RF pulse sequence of the TOF sequence in FIG. 2 has two parts: a first TOF subsequence 210 and a second TOF subsequence 220 (the first part and the last part separated by a dotted line in FIG. 2). The first TOF subsequence 210 has four T-sat modules 211 and four excitation modules 212, and a T-sat module 211 is directly followed by an excitation module 212. The second TOF subsequence 220 has only four excitation modules 222. That is to say, the second TOF subsequence has no T-sat module. In FIG. 2, the t axis represents the time axis.

FIG. 3 is a schematic diagram for a TOF pulse sequence according to another embodiment of the present application. As shown in FIG. 3, the TOF sequence in FIG. 3 has two parts: a first TOF subsequence 310 and a second TOF subsequence 320 (the first part and the last part separated by a dotted line in FIG. 3). The first TOF subsequence 310 has two T-sat modules 311 and four excitation modules 312, and a T-sat module 311 is directly followed by two excitation modules 312. The second TOF subsequence 320 has only four excitation modules 322, and no T-sat module. In FIG. 3, the t axis again represents the time axis.

It should be noted that for the convenience of a simpler representation, only T-sat RF pulses are used to represent T-sat modules and excitation RF pulses are used to represent excitation modules, but the gradient pulses contained in T-sat modules and excitation modules are not shown in FIG. 2 and FIG. 3. In addition, FIG. 2 and FIG. 3 are only two examples of TOF pulse sequences, and the TOF sequences in the present application are not limited to these two examples.

Step 102: Acquire k-space data as a first k-space data portion after each excitation module of said first TOF subsequence.

The magnetic resonance signals acquired by the reception coil of a magnetic resonance system are analog RF signals containing spatially encoded information. Therefore, the magnetic resonance analog signals need to be converted into digital information through an analog-digital conversion (ADC), and are then entered in k-space. In the embodiments of the present application, the digital information obtained from the magnetic resonance signals acquired by the reception coil after an ADC is called k-space data.

In the embodiments of the present application, after each excitation module of the first TOF subsequence, the coil will acquire magnetic resonance analog signals and the magnetic resonance analog signals are converted into a first k-space data portion after an ADC.

Step 103: Fill the first k-space data portion in the central area of k-space.

In the embodiments of the present application, the entirety of k-space can be divided into two parts: a central area and an edge (peripheral) area. Those skilled in the art know that the central area of k-space mainly affects the contrast of magnetic resonance imaging, while the edge area of k-space mainly affects the details of magnetic resonance imaging. That is to say, the central area and edge area of k-space affect the magnetic resonance imaging effect differently.

In addition, in the embodiments of the present application, the percentage of the central area of k-space to the entire area of k-space is called the percentage of the central area of k-space, and in order to adjust the magnetic resonance imaging effect and the scanning time, the percentage of the central area of k-space can be set and adjusted, for example, usually to 25% to 100%, preferably 50% to 75%.

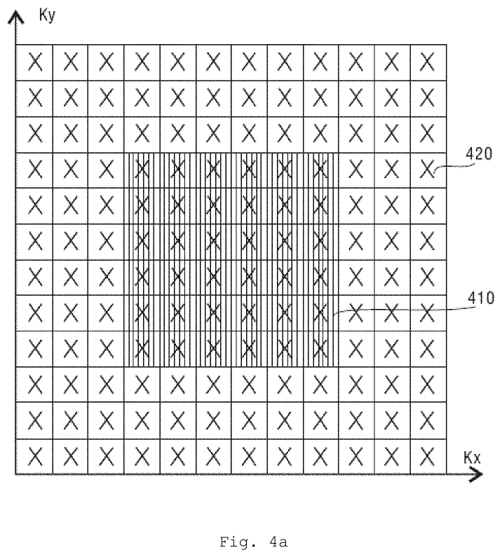

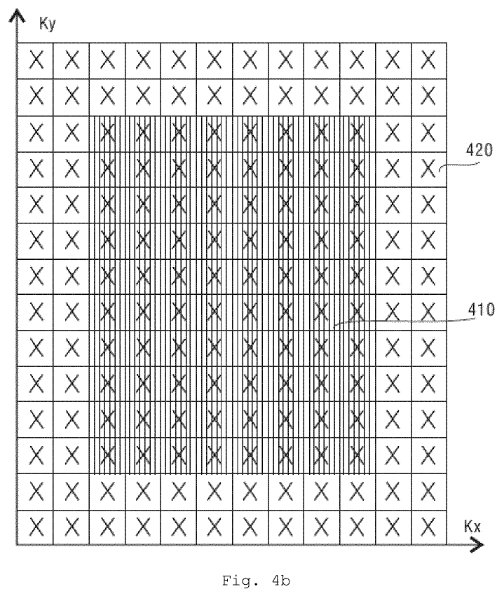

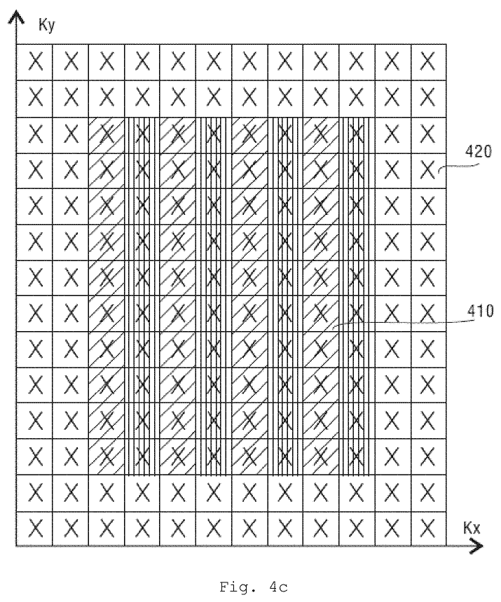

FIG. 4a is a schematic diagram of k-space according to one embodiment of the present application. FIG. 4a shows an example of the division of the whole area of k-space into a central area and an edge area. In FIG. 4a, each small check represents a readout, and Kx and Ky respectively represent the two coordinate axes of k-space. The 36 shaded small checks in FIG. 4a represent the central area 410 of k-space, and the other small checks represent the edge area 420 of k-space. The percentage of the central area of k-space is about 25% in the k-space division shown in FIG. 4a.

FIG. 4b is a schematic diagram of k-space according to another embodiment of the present application. FIG. 4b shows another example of the division of the whole area of k-space into a central area and an edge area. Similar to FIG. 4a, in FIG. 4b, each small check represents a readout, and Kx and Ky respectively represent the two coordinate axes of k-space. The 80 shaded small checks in FIG. 4b represent the central area 410 of k-space, and the other small checks represent the edge area 420 of k-space. The percentage of the central area of k-space is about 50% in the k-space division shown in FIG. 4b.

It should be noted that FIG. 4a and FIG. 4b only give examples of the division of k-space into the central area and the edge area, but the division of k-space in the present application is not limited to the forms and percentages shown in FIG. 4a and FIG. 4b. For example, the central area 410 in FIG. 4a and FIG. 4b is in a square shape, but the central area in the present invention is not limited to being a square shape and can be in a round shape or in other shapes. In addition, the central area and edge area of k-space are also relative to the data sizes of all k-space data filled in the whole k-space in a magnetic resonance scanning, and are relative concepts, and the number of readouts included in the central area or edge area is not limited in the present invention.

From Step 102 and Step 103, it can be seen that the first TOF subsequence comprises at least a T-sat module, and through the excitation of T-sat RF pulses, the acquired k-space data can effectively suppress the signals of venous blood flowing into the angiographic plane to obtain a good imaging effect. In addition, as described previously, the central area of k-space mainly affects the contrast of magnetic resonance imaging. Therefore, in the embodiments of the present application, the filling of the acquired first k-space data portion in the central area of k-space can guarantee that the final imaging result has good contrast.

Step 104: Acquire a second k-space data portion after each excitation module of the second TOF subsequence.

Similarly, in the embodiments of the present application, the reception coil will acquire magnetic resonance analog signals after each excitation module of the second TOF subsequence. In addition, the magnetic resonance analog signals are converted into a second k-space data portion after an ADC.

Step 105: Fill the second k-space data portion in the edge area of said k-space.

From Step 104 and Step 105, it can be seen that the second TOF subsequence comprises only excitation modules but does not comprise any T-sat modules. Since the power of T-sat RF pulses in T-sat modules is higher than that of excitation RF pulses in excitation modules and the duration of T-sat RF pulses in T-sat modules is much longer than that of excitation RF pulses in excitation modules, the scanning time can be shortened greatly and the SAR can be lowered, although the acquired k-space data cannot effectively suppress the signals of venous blood flowing into the angiographic plane during angiography and the imaging effect is lost. In addition, as described previously, the edge area of k-space mainly affects the details of magnetic resonance imaging. Therefore, in the embodiments of the present application, the filling of the acquired second k-space data portion in the edge area of k-space affects only the details of the imaging result, instead of the contrast of the imaging result, that is to say, affects the imaging result little, compared with the central area.

Step 106: Use the k-space data after filling to reconstruct a magnetic resonance image.

After k-space data acquisition and filling from Step 102 to Step 105, complete k-space data can be obtained, and a magnetic resonance image can be reconstructed according to the filled k-space data.

As described previously, with the inventive method, k-space is divided into a central area and an edge area. For the central area, which affects the imaging result greatly, k-space data excited by a TOF sequence including T-sat modules are acquired so as to assure good image quality, and for the edge area, which affects the imaging result less, k-space data excited by a TOF sequence having no T-sat module are acquired so as to reduce the total number of T-sat modules used for a TOF sequence. Thus, the scanning time is reduced accordingly, the scanning efficiency is improved, and the SAR is lowered.

In addition, in the embodiments of the present application, the percentage of the central area of k-space can be set, and the ratio of the number of excitation modules of the first TOF subsequence of a TOF sequence to the number of excitation modules of the second TOF subsequence can be controlled according to the percentage of the central area of k-space so that a balance can be found between the imaging effect and the scanning time/SAR. To be specific, the ratio of the number of excitation modules of the first TOF subsequence to the total number of excitation modules of the TOF sequence can be equal to the percentage of the central area of k-space. For example, if the percentage of the central area of k-space is 50% and the TOF sequence comprises eight excitation modules, then the first TOF subsequence comprises four excitation modules and the second TOF subsequence also has four excitation modules. This is also shown by the examples in FIG. 2 and FIG. 3.

In the embodiments of the present application, said percentage of the central area of k-space can be set according to the experience and can vary with detected parts and detected persons. In addition, the percentage of the central area of k-space is adjustable so that a tradeoff is obtained between the reduction of the image effect and the reductions of the scanning time and the SAR. Thus, while the imaging effect is assured, the percentage of the central area of k-space is reduced as much as possible so as to reduce the scanning time and the SAR as much as possible. If the percentage of the central area of k-space is large, the imaging result approaches that of a conventional TOF sequence, i.e., the imaging result is good. If the percentage of the central area of k-space is small, the scanning time is short and the SAR is low.

In embodiments of the present application, a T-sat module of the first TOF subsequence can directly be followed by an excitation module or can directly be followed by a plurality of excitation modules.

In an embodiment of the present application, the TOF sequence segmentation value can further be set. The TOF sequence segmentation value can be used to control the number of excitation modules directly following a T-sat module of said first TOF subsequence. If the TOF sequence segmentation value is N, wherein N is a positive integer, a T-sat module of the first TOF subsequence can directly be followed by N excitation modules. Through such a TOF sequence segmentation setting, the number of T-sat modules of the first TOF subsequence can further be reduced. Thus, the scanning time is further shortened and the SAR is lowered.

In the example shown in FIG. 3, N=2. This means that each T-sat module is followed by two excitation modules. As shown in FIG. 3, the first TOF subsequence comprises only two T-sat modules and four excitation modules. Compared with the TOF sequence shown in FIG. 2, the number of T-sat modules is further reduced by two. Thus, compared with the solution in FIG. 2, the scanning time can further be shortened (it can also be seen from the figures that it takes a shorter time to scan the TOF pulse sequence in FIG. 3 than to scan the TOF pulse sequence in FIG. 2) and the SAR can be reduced.

FIG. 4c is a schematic diagram of k-space according to a further embodiment of the present application. FIG. 4c shows an example of the division of the whole area of k-space into a central area and an edge area, with a TOF sequence segmentation value of 2. Similar to FIG. 4a and FIG. 4b, in FIG. 4c, each small check represents a readout, and Kx and Ky respectively represent the two coordinate axes of k-space. The 40 small checks with slashes in FIG. 4c represent the k-space data read out and filled after the excitation of the excitation modules following a T-sat module, and the 40 small checks with vertical lines in FIG. 4c represent the k-space data that were read out and filled after the excitation of the excitation modules far away from a T-sat module. The readouts of these two portions are filled in the central area 410 of k-space, and the other small checks represent the edge area 420 of k-space. The percentage of the central area of k-space is about 50% and the TOF sequence segmentation value is 2 in the k-space division shown in FIG. 4c. Of course, without loss of generality, the 40 small checks with slashes in FIG. 4c can also represent the k-space data read out and filled after the excitation of the excitation modules far away from a T-sat module, and the 40 small checks with vertical lines in FIG. 4c can also represent the k-space data read out and filled after the excitation of the excitation modules following a T-sat module.

It should be noted that the central area 410 in FIG. 4c has a square shape, but the central area in the present invention is not limited to being a square shape and can be a round shape or other shapes.

In embodiments of the present application, the TOF sequence segmentation value N is adjustable. Wherein, the larger N is, the more the scanning time and the SAR are reduced. Of course, the setting of N also affects the imaging effect. The smaller N is, the better imaging effect. Therefore, a tradeoff can also be obtained between the reduction of the imaging effect and the reductions of the scanning time and the SAR by adjusting N. Thus, while the imaging effect is assured, the percentage of the central area of k-space is reduced as much as possible so as to reduce the scanning time and the SAR as much as possible.

As an alternative solution of the above-mentioned solution, other rapid k-space data acquisition techniques can be combined with the technical solution in the present application to further shorten the scanning time and lower the SAR.

In embodiments of the present application, known rapid parallel acquisition techniques can be combined for the acquisition of k-space data. Acquiring k-space data after each excitation module of the first TOF subsequence in Step 102 of the method for MRA can be done using the rapid parallel acquisition technique to acquire the k-space data after each excitation module of the first TOF subsequence. Acquiring k-space data after each excitation module of the second TOF subsequence in Step 104 also can be done using a rapid parallel acquisition technique to acquire k-space data after each excitation module of the second TOF subsequence.

Such rapid parallel acquisition technique can comprise various accelerated imaging techniques, for example, parallel acquisition technique used in the image reconstruction, parallel acquisition technique used for k-space filling, and controlled aliasing parallel imaging results in higher acceleration (CAIPIRINHA) acquisition technique.

The use of such rapid parallel acquisition technique for k-space data acquisition can further shorten the time necessary for acquiring k-space data, and thereby shorten the scanning time, improve the imaging efficiency, and reduce the SAR is achieved.

The present application also encompasses an apparatus for MRA. FIG. 5 is a block diagram of the basic components of the MRA apparatus according to an embodiment of the present application. As shown in FIG. 5, the MRA apparatus has an MRA data acquisition scanner 501, a computer that operates the scanner 501, serving as an acquisition module 502 and a filling module 503, and an image reconstruction processor 504.

The scanning module 501 is operated by the computer so as to execute a TOF sequence scan.

In the embodiment of the present application, the TOF sequence includes a first TOF subsequence and a second TOF subsequence. The first TOF subsequence has at least a T-sat module and at least an excitation module, with a T-sat module is directly followed by at least an excitation module. The second TOF subsequence has at least an excitation module and no T-sat module.

The computer serving as the acquisition module 502 is configured to operate the scanner 501 in order to acquire k-space data as a first k-space data portion after each excitation module of the first TOF subsequence, and to acquire k-space data as a second k-space data portion after each excitation module of the second TOF subsequence.

In the embodiments of the present application, the acquisition module acquires magnetic resonance analog signals after each excitation module of the first TOF subsequence and converts the magnetic resonance analog signals into a first k-space data portion after an ADC. The acquisition module acquires magnetic resonance analog signals after each excitation module of the second TOF subsequence and converts the magnetic resonance analog signals into a second k-space data portion after an ADC.

The computer serving as the filling module 503 is configured to fill the first k-space data portion in the central area of k-space and fill the second k-space data portion in the edge area of k-space.

The image reconstruction processor 504 is configured to use the k-space data after filling to reconstruct a magnetic resonance image.

In the embodiments of the present invention, said apparatus can further include a setting module formed by the computer, configured to set a percentage of the central area of k-space, wherein the ratio of the number of excitation modules of the first TOF subsequence to the total number of excitation modules of the TOF sequence is equal to the percentage of the central area of k-space. The percentage of the central area of k-space is adjustable.

In the embodiments of the present invention, the setting module can further be configured to set a TOF sequence segmentation value N, wherein a T-sat module of the first TOF subsequence is directly followed by N excitation modules and N is a positive integer. The TOF sequence segmentation value N is also adjustable.

To further accelerate scanning, the acquisition module 502 can use a rapid parallel acquisition technique to acquire the k-space data. For example, the acquisition module 502 can use a parallel acquisition technique for image reconstruction, a parallel acquisition technique for k-space filling, or the CAIPIRINHA acquisition technique.

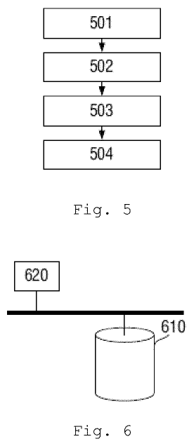

As shown in FIG. 6, the MRA apparatus has at least one memory 610, and at least one processor 620 of the computer. The at least one memory 610 stores a computer program, and the at least one processor 620 is configured to access the computer program stored in said at least one memory to execute the inventive method for MRA.

Alternatively, the computer program can be distributed among the modules of the computer shown in FIG. 5.

In addition, THE at least one memory 610 can store an operating system. The operating system includes but is not limited to Android operating system, Symbian operating system, Windows operating system and Linux operating system.

The at least one processor 620 is configured to access the computer program stored in at least one memory 610 in order to execute the method in the embodiments of the present invention, with at least one data-receiving port. The processor 620 can be a central processing unit (CPU), a processing unit/module, an application specific integrated circuit (ASIC), a logic module or a programmable array.

It should be noted that not all the steps in the flowchart in FIG. 1 or the modules in the schematic diagram for the structure of the device for MRA in FIG. 5 are necessary, and some steps or modules can be ignored according to the actual requirements. The execution sequence of the steps is not fixed and can be adjusted as required. The partition of the modules is a functional partition for the convenience of description. In the practical implementation, the function of a module can be realized by a number of modules, and the functions of a plurality of modules can be realized by one module and these modules can be located in the same equipment or can be located in different equipment.

The hardware modules in different embodiments can mechanically or electronically be realized. For example, a hardware module can comprise specially designed permanent circuits or logic devices (for example, application-specific processors such as field programmable gate array (FPGA) or ASIC) to complete specific operations. A hardware module can also comprise programmable logic devices or circuits (for example, general processors or other programmable processors) temporarily configured by software to perform specific operations. Whether a hardware module is realized mechanically, or by use of a dedicated permanent circuit or a temporarily configured circuit (for example, configured by software) can depend on the considerations of the cost and the time.

The present invention further encompasses a machine readable storage medium (for example, computer readable storage medium), which stores instructions used to enable a machine to execute the method in the present application. Specifically, a system or device equipped with a storage medium can be provided. Software program codes which can realize the function in any of above-mentioned embodiments are stored in the storage medium and the computer (or CPU or MPU) of the system or device can read out and execute the program codes stored in the storage medium. In addition, through the instructions based on the program codes, the operating system on the computer can complete a part of or all of practical operations. In addition, the program codes read out of a storage medium can be written into the memory in the expansion board in a computer or can be written into a memory in an expansion unit connected to the computer, and then the instructions based on the program codes let the CPU installed on the expansion board or expansion unit execute a part or all of practical operations to realize the function in any of the above-mentioned embodiments. Storage media used to provide program codes include floppy disk, hard disk, magneto-optical disk, compact disk (for example, compact disk read-only memory (CD-ROM)), compact disk—recordable (CD-R), compact disk—rewritable (CD-RW), digital video disk—read only memory (DVD-ROM), digital versatile disk—random access memory (DVD-RAM), digital versatile disk—rewritable (DVD-RW), magnetic tape, non-volatile memory card, and read-only memory (ROM). Alternatively, the program codes can be downloaded from the server computer over a communication network.

As described previously, in the inventive MRA apparatus, k-space is divided into a central area and an edge area, wherein for the central area that affects the imaging result greatly, k-space data excited by a TOF sequence having T-sat modules are acquired so as to assure a good image quality, and for the edge area which affects the imaging result less, k-space data excited by a TOF sequence having no T-sat module are acquired so as to reduce the total number of T-sat modules used for a TOF sequence. Thus, the scanning time is reduced accordingly, the scanning efficiency is improved, and the SAR is reduced.

In addition, a T-sat module of the first TOF subsequence can directly be followed by an excitation module or can directly be followed by a number of excitation modules. When a T-sat module of the first TOF subsequence is directly followed by a number of excitation modules, the number of T-sat modules of the first TOF subsequence can further be reduced. Thus, the scanning time is further shortened and the SAR is reduced.

In addition, the use of a rapid parallel acquisition technique for k-space data acquisition can further shorten the time necessary for acquiring k-space data, and thus shorten the scanning time, improve the imaging efficiency, and reduce the SAR is achieved.

The following further describes by experiment the effect of the technical solution provided by the embodiments of the present application. In the experiment, the person under test wore an a16-channel head/neck coil and a Siemens free-breathing 1.5T magnetic resonance imaging system MAGNETOM Amira was used for MRA.

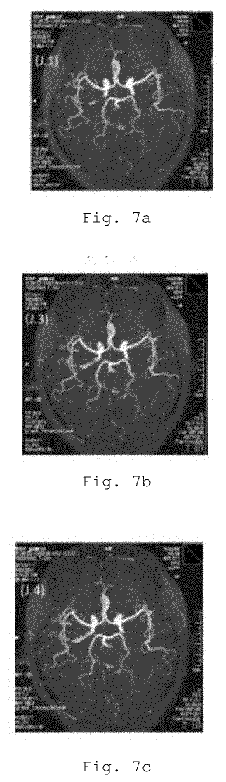

FIG. 7a shows the imaging result produced by scanning a conventional TOF sequence. FIG. 7b shows one imaging result formed by using the method of the present application, wherein the percentage of the central area of k-space is 50% and N=1. FIG. 7c shows another imaging result formed by using the method of the present application, wherein the percentage of the central area of k-space is 50% and N=2. Wherein, the scanning time necessary for obtaining the image shown in FIG. 7a is 5 minutes and 11 seconds, the scanning time necessary for obtaining the image shown in FIG. 7b is 4 minutes and 21 seconds, and the scanning time necessary for obtaining the image shown in FIG. 7c is 3 minutes and 56 seconds. From FIG. 7a, FIG. 7b and FIG. 7c, it can be seen that the imaging effects differ little, but the scanning time used in FIG. 7c is reduced by over 24% than the scanning time used in FIG. 7a. That is to say, the scanning time is greatly shortened. In addition, since the number of T-sat modules is reduced by 75%, the SAR is greatly lowered. Thus, it can be seen that the method for MRA provided in the present application can achieve a very good imaging effect, greatly shorten the scanning time and lower the SAR while improving the imaging efficiency.

It should be understood that although the Description gives a description by embodiment, it does not mean that each embodiment contains only one independent technical solution. The description method in the Description is only for the sake of clarity. Those skilled in the art should consider the Description as an integral body. The technical solutions in all these embodiments can be combined properly to form other embodiments that those skilled in the art can understand.

The series of detailed descriptions above are only specific descriptions of feasible embodiments of the present invention and they are not intended to limit the scope of protection of the present invention. All equivalent embodiments or variants, for example, combination, division, or duplication of technical characteristics are considered to be within the scope of the present invention.

1. A method for magnetic resonance angiography (MRA), comprising:
operating a magnetic resonance data acquisition scanner in order to execute a time of flight (TOF) MRA data acquisition sequence comprising a first TOF subsequence comprising at least a T-sat module and at least an excitation module, with said T-sat module being directly followed by at least an excitation module, and said TOF sequence also comprising a second TOF subsequence comprising at least an excitation module and no T-sat module;
in a computer, converting the acquired MRA data into k-space data comprising a first k-space data portion acquired after each excitation module of said first TOF subsequence, and a second k-space data portion acquired after each excitation module of said second TOF subsequence;
entering said first k-space data portion into a central area of k-space in a memory organized as k-space, and entering said second k-space data portion into an edge area of k-space in said memory organized as k-space; and
reconstructing magnetic resonance angiography image data from said k-space data entered into said memory organized as k-space.

2. A method as claimed in claim 1 comprising, in said computer, setting a percentage of the central area of k-space to an entire area of k-space, and setting a ratio of a number of said excitation modules of said first TOF subsequence to a total number of excitation modules of said TOF sequence is equal to said percentage.

3. A method as claimed in claim 2 comprising selecting said percentage so as to produce a selected tradeoff between a quality of said MRA image data and a time duration required to execute said TOF sequence.

4. A method as claimed in claim 1 comprising, in said computer, setting a positive integer segmentation value N that causes said T-sat module in said first TOF subsequence to be directly followed by N excitation modules in said first TOF subsequence.

5. A method as claimed in claim 2 comprising selecting N so as to produce a selected tradeoff between a quality of said MRA image data and a time duration required to execute said TOF sequence.

6. A method as claimed in claim 1 comprising acquiring said k-space data after each excitation in each of said first and second TOF subsequences by executing a rapid parallel acquisition technique in said magnetic resonance data acquisition scanner.

7. A magnetic resonance angiography (MRA) apparatus comprising:
a magnetic resonance data acquisition scanner;
a computer configured to operate said magnetic resonance data acquisition scanner in order to execute a time of flight (TOF) MRA data acquisition sequence comprising a first TOF subsequence comprising at least a T-sat module and at least an excitation module, with said T-sat module being directly followed by at least an excitation module, and said TOF sequence also comprising a second TOF subsequence comprising at least an excitation module and no T-sat module;
said computer being configured to convert the acquired MRA data into k-space data comprising a first k-space data portion acquired after each excitation module of said first TOF subsequence, and a second k-space data portion acquired after each excitation module of said second TOF subsequence;
said computer being configured to enter said first k-space data portion into a central area of k-space in a memory organized as k-space, and entering said second k-space data portion into an edge area of k-space in said memory organized as k-space; and
said computer being configured to reconstruct magnetic resonance angiography image data from said k-space data entered into said memory organized as k-space.

8. An apparatus as claimed in claim 7 wherein said computer is configured to set a percentage of the central area of k-space to an entire area of k-space, and to set a ratio of a number of said excitation modules of said first TOF subsequence to a total number of excitation modules of said TOF sequence is equal to said percentage.

9. An apparatus as claimed in claim 7 wherein said computer is configured to set a positive integer segmentation value N that causes said T-sat module in said first TOF subsequence to be directly followed by N excitation modules in said first TOF subsequence.

10. An apparatus as claimed in claim 7 wherein said computer is configured to operate said magnetic resonance data acquisition scanner in order to acquire said k-space data after each excitation in each of said first and second TOF subsequences by executing a rapid parallel acquisition technique in said magnetic resonance data acquisition scanner.

11. A non-transitory, computer-readable data storage medium encoded with programming instructions, said storage medium being loaded into a computer of a magnetic resonance angiography (MRA) apparatus comprising a magnetic resonance data acquisition scanner, said programming instructions causing said computer to:
operate the magnetic resonance data acquisition scanner in order to execute a time of flight (TOF) MRA data acquisition sequence comprising a first TOF subsequence comprising at least a T-sat module and at least an excitation module, with said T-sat module being directly followed by at least an excitation module, and said TOF sequence also comprising a second TOF subsequence comprising at least an excitation module and no T-sat module;
convert the acquired MRA data into k-space data comprising a first k-space data portion acquired after each excitation module of said first TOF subsequence, and a second k-space data portion acquired after each excitation module of said second TOF subsequence;
enter said first k-space data portion into a central area of k-space in a memory organized as k-space, and entering said second k-space data portion into an edge area of k-space in said memory organized as k-space; and
reconstruct magnetic resonance angiography image data from said k-space data entered into said memory organized as k-space.
